(19) United States
(12) Patent Application Publication
(10) Pub. No.: US 2007/0275376 A1
(43) Pub. Date: Nov. 29, 2007

(54) Tumor-Specific Expression of Reporter Genes

(76) Inventors: Joerg Heyer, Cambridge, MA (US); Ronan C. O'Hagan, Arlington, MA (US)

Correspondence Address: ROPES & GRAY LLP;PATENT DOCKETING 39/361, 1211 AVENUE OF THE AMERICAS, NEW YORK, NY, 10036-8704, US

(21) Appl. No.: 10/570,117

(22) PCT Filed: Aug. 27, 2004
(86) PCT No.: PCT/US04/27970

§ 371 Date: Dec. 12, 2006

Related U.S. Application Data
(60) Provisional application No. 60/499,310, filed on Aug. 28, 2003.

(51) Int. Cl. A01K 67/027; C12Q 1/68; C07H 21/04; C12N 5/08; C12N 5/06

(52) U.S. Cl. 435/6; 435/325; 536/23.1; 536/23.2; 800/14; 800/18; 435/354; 435/366

(57) ABSTRACT

A reporter gene operably linked to a transcriptional control element that is up-regulated by a tumor-induced transcription factor is disclosed. This reporter gene construct can be used to detect tumor formation in vivo in an experimental animal.

### FIELD OF THE INVENTION

The field of the invention is molecular biology, embryology, bio-imaging and cancer biology.

### BACKGROUND OF THE INVENTION

Tumors develop from normal cells in a tissue by acquiring mutations in their genome that help to overcome the growth restriction in the tissue. Imaging developing tumors is a challenging task due to the fact that the initial tumor mass is very small compared to the normal tissue mass. The existing method for visualizing the development of tumors uses tissue-specific expression of a reporter gene (e.g., Vooijs et al., Cancer Res. 62(6): 1862-7 (2002)). Because all cells in the tissue express the reporter, only larger tumor masses can be seen in imaging. Early stage, small tumors may go undetected.

### SUMMARY OF THE INVENTION

The invention provides a gene construct containing a reporter gene operably linked to a promoter containing a transcriptional regulatory element that is up-regulated by a transcription factor preferentially produced in neoplastic cells. Exemplary transcriptional regulatory elements are a β-catenin response element, an E2F response element, a Forkhead response element, and a Smad-2/Smad-3 response element. The reporter gene encodes a protein such as an enzyme, a bioluminescent protein, or a fluorescent protein. Examples of enzymes useful as a reporter gene are β-galactosidase, alkaline phosphatase, and chloramphenicol acetyltransferase. An exemplary bioluminescent protein is a luciferase. Useful fluorescent proteins include green fluorescent protein, yellow fluorescent protein, enhanced yellow fluorescent protein, red fluorescent protein and blue fluorescent protein.

The invention also provides a cell comprising the above-described gene construct. Preferably, the cell also contains a neoplastic transformation-promoting genetic modification. The cell can be, e.g., an embryonic stem (ES) cell.

The invention also provides a nonhuman mammal, e.g., a mouse, that contains a cell that contains a recombinant gene construct containing a reporter gene operably linked to a promoter containing a transcriptional regulatory element that is up-regulated by a transcription factor preferentially produced in neoplastic cells. Preferably, the nonhuman mammal also contains a neoplastic transformation-promoting genetic modification.

The invention also provides a method of detecting neoplasia in a nonhuman mammal. The method includes the steps of (a) providing a nonhuman mammal, e.g., a mouse, at least some of whose somatic cells are engineered cells containing a genome that includes (i) a neoplastic transformation-promoting genetic modification and (ii) a reporter gene operably linked to a promoter containing a transcriptional regulatory element that is up-regulated by a transcription factor preferentially produced in neoplastic cells; (b) allowing time for neoplastic transformation to occur in at least one of the engineered cells, and (c) detecting a signal from the reporter gene expressed in the engineered cells.

This invention allows highly sensitive detection of small, developing tumors that are otherwise undetected by existing methods. Additionally, the invention offers the advantage of decreasing the number of experimental animals and the tumor load in each animal needed to screen potential therapeutic compounds.

Other features and advantages of the invention will be apparent from the following detailed description.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. The materials, methods, and examples are illustrative only and not intended to be limiting. Throughout this specification and claims, the word “comprise,” or variations such as “comprises” or “comprising” will be understood to imply the inclusion of a stated integer or group of integers but not the exclusion of any other integer or group of integers.

### DETAILED DESCRIPTION OF THE INVENTION

Tumor development often affects key signaling pathways in a tumor cell, resulting in increased activities of one or more transcription factors controlled by these pathways in the tumor cell as compared to a normal cell. This invention features a reporter gene placed under the control of a transcriptional regulatory element that is up-regulated by such a tumor-induced transcription factor. A tumor cell expresses the reporter gene at a higher level than its normal counterpart. Thus, the reporter gene can be used to detect tumors in live animals at any stage of tumor development, e.g., during early stages of tumorigenesis, even when the tumor is too small to be detected by convention methods.

Methods of the invention can be used to study the development of cancer in an objective, real time, quantitative and noninvasive manner. More particularly, the invention facilitates the study of cancer initiation, progression, maintenance, metastasis, regression, minimal residual disease, and recurrence. Typically, the invention is incorporated into a nonhuman mammal, e.g., a mouse, that is engineered to serve as an in vivo model of a particular type of cancer. This approach to tumor imaging is a significant advance for rapid and dynamic screening as well as validation of experimental therapeutic agents.

Tumor-Specific Reporter Genes

The reporter gene of this invention is operably linked to a transcriptional regulatory element, e.g., a promoter or enhancer, that is up-regulated by a tumor-induced transcription factor. Consequently, the reporter gene will have basal expression levels in a normal tissue and higher expression levels in a tumor tissue. As used herein, “transcription factor,” means a protein involved in the transcription of genes. “Tumor-induced transcription factors” are those whose expression or activity is increased during tumor development.

An example of a tumor-induced transcription factor is β-catenin, which regulates cell proliferation and differentiation in the mucosal membrane where gastrointestinal tumors typically arise. β-catenin is normally sequestered in the cytoplasm by the Adenomatous Polyposis Coli (APC) protein. APC is a gastrointestinal tumor suppressor, the inactivation of which (e.g., due to deletion of the APC gene) is a hallmark of many gastrointestinal tumors, including colon cancer. When APC is inactivated, β-catenin relocates to the nucleus and forms a transcription factor complex with T Cell Factor 4 (TCF4) and other transcription factors. The transcription factor complex binds to promoters that contain (are operatively linked to) a β-catenin response element, resulting in expression of genes controlled by these promoters. Examples of such genes are those encoding c-myc, CD44, BMP4, CLAUDIN I, Cyclin D1, FRA1, NrCAM, PKD1, Survivin, and Ephrin B (Giles et al., Biochimica et Biophysica Acta 1653:1-24 (2003); Kim et al., Lancet 362:205-9 (2003)). Thus, expression of a reporter gene operably linked to a promoter controlled by the β-catenin transcription factor complex, such as the promoter of one of the aforementioned genes or an artificial Top/Fop promoter (see below), indicates tumor formation in the gastrointestinal tissue.

β-catenin is also activated in hepatocarcinoma caused by loss of Axin activity. Other examples of tumor-induced transcription factors are Forkhead, which is activated in prostate cancer caused by loss of PTEN activity; and Smad-2 and Smad-3, which are activated in pancreatic cancer caused by loss of Smad-4 or DPC-4 activity. A reporter gene linked to a transcriptional regulatory element up-regulated by one of these transcription factors is useful in monitoring tumors in the liver, prostate gland, and pancreas, respectively.

When expressed in a cell, a reporter gene of this invention produces a detectable phenotypic change in the cell. The reporter gene may encode a product whose activity is not normally found in the organism of interest and thus may be easily assayed, or encode a product that is naturally found in the organism of interest but not naturally found in the tissue that gives rise to the tumor. Useful reporter genes include those encoding enzymes, enzymatic substrates, luminescent proteins, and fluorescent proteins, such as luciferase, β-galactosidase, alkaline phosphatase, chloramphenicol acetyltransferase, green-fluorescent protein (GFP), and variants of GFP. Assays for reporter genes are known in the art. Reporter genes, assay kits and other materials are available commercially, e.g., from Promega Corp. (Madison, Wis.) and GIBCO BRL (Gaithersburg, Md.).

In some embodiments, a gene encoding luciferase or an equivalent thereof is used. Members of the luciferase family have been identified in a variety of prokaryotic and eukaryotic organisms. Luciferase and other enzymes involved in the prokaryotic luminescence systems, as well as the corresponding lux genes, have been isolated from marine bacteria in the Vibrio and Photobacterium genera and from terrestrial bacteria in the Xenorhabdus genus. Eukaryotic organisms (e.g., firefly) also can be used to obtain luciferases.

In other embodiments, a gene encoding green fluorescent protein (GFP) is used. GFP is a fluorescent protein isolated from coelenterates such as the Pacific jellyfish Aequoria victoria. There are many useful variants of GFP that can also be used in this invention. They include, without limitation, enhanced GFP (EGFP), yellow fluorescent protein, red fluorescent protein, blue fluorescent protein, etc. Constructs encoding these fluorescent proteins are available commercially, e.g., from Amersham.

The reporter gene of the invention can be inserted into a vector such as a viral vector, a plasmid vector or an artificial chromosome. Examples of retroviral vectors include murine leukemia viral vectors, adenovirus vectors, herpes virus vectors, and lentiviral vectors. Examples of artificial chromosomes include a BAC, a PAC, a YAC or a MAC. These vectors can be introduced into a host cell, e.g., an oocyte, a embryonic or tissue-specific stem cell, or a differentiated cell, by infection, microinjection, transfection, transposome/liposome fusion, and the like.

Use of Tumor-Specific Reporter Genes

The tumor-specific reporter genes of this invention can be used to detect tumors in vivo. The reporter gene can be introduced into a pre-malignant cell (or an ancestor thereof) in an animal. Once the cell becomes malignant, the expression or activity of a tumor-induced transcription factor is increased. This up-regulates the transcriptional regulatory element in the promoter operably linked to the reporter gene. This results in increased expression of the reporter gene, thereby enabling one to detect tumors in vivo, even when the tumors are too small to be detected by conventional methods such as palpation.

Tumors in the animal can arise spontaneously or can be induced artificially de novo. Tumorigenesis can be induced by methods known in the art, e.g., by exposure to carcinogens or exposure to UV radiation or gamma radiation. Alternatively, the animal can contain cells whose genomes comprise a genetic modification that predisposes the cells to neoplastic transformation, i.e., a neoplastic transformation-promoting genetic modification.

In some embodiments, neoplastic transformation-promoting genetic modification may be an introduced oncogene under inducible transcriptional control. Expression of the oncogene can be induced by, e.g., a Cre-Lox system and any of the inducible transcription systems for RNA polymerase II (e.g., the tetracycline transactivator systems, reverse tetracycline transactivator systems, ecdysone systems, methallothionine systems, LacO/IPTG systems, and TetO/tetracycline systens). See, e.g., WO 01/09308. Inducible transcription systems for RNA polymerases I and III can also be used with or without modifications.

A neoplastic transformation-promoting genetic modification also can be a disabling (e.g., null, conditionally null, or dominant negative) mutation in a tumor suppressor gene (e.g., INK4a, P53, APC, PTEN, Rb (Jacks et al., Nature 359:295-300(1992), DPC4, KLF6, GSTP1, ELAC2/HPC2, or NKX3.1). It also can be a deletion or disabling mutation in a DNA repair gene (e.g., MSH2, MSH6, PMS2, Ku70, Ku80, DNA/PK, ATR, ATM, XRCC4, or MLH1). It also can be an activating mutation in an endogenous proto-oncogene (e.g., myc and ras). Such genetic modifications can be introduced into the genome of a host cell by conventional homologous recombination technologies (e.g., gene knock out or knock in).

Expression of a tumor suppressor gene or a DNA repair gene can be reduced by RNA interference (RNAi) constructs introduced into the host genome. RNAi is a sequence-specific posttranscriptional gene silencing mechanism triggered by double-stranded RNA (dsRNA). It causes degradation of mRNAs homologous in sequence to the dsRNA. See, e.g., Elbashir et al., Methods 26:199-213 (2002); McManus and Sharp, Nature Reviews 3:737-747 (2002); Hannon, Nature 418:244-251 (2002); Brummelkamp et al., Science 296:550-553 (2002); Tuschl, Nature Biotechnology 20:446-448 (2002); U.S. Pat. No. 6,506,559; U.S. Application US2002/0086356 A1; WO 99/32619; WO 01/36646; and WO 01/68836.

Preferably, the reporter gene and the neoplastic transformation-promoting genetic modification(s) are in the same cells, so the reporter gene indicates only tumor formation caused by the genetic modification. This can be achieved, e.g., by introducing into a host cell separate vectors, one containing the reporter gene and the other containing the neoplastic transformation-promoting genetic modification(s). This also can be achieved by introducing into a host cell a single vector that contains both the reporter gene and the neoplastic transformation-promoting genetic modification(s). In some embodiments, the cell contains more than one neoplastic transformation-promoting genetic modification. For example, the cell may contain an inducible oncogene and a null mutation in an endogenous tumor suppressor gene.

The vectors may be administered into host cells by various methods, including but not limited to, liposome/transposome fusion, routine nucleic acid transfection methods such as electroporation, calcium phosphate precipitation, and microinjection, and infection by viral vectors. The vectors may be introduced into host cells in an animal in vivo or ex vivo. For instance, the vectors may be administered to any tissue in an animal, including without limitation: dermal, brain, heart, lung, kidney, colon, gastrointestinal, prostate, ovarian, breast, liver or bone tissue. The vectors can also be introduced into tissue-specific stem cells (including progenitor cells) ex vivo, which are then implanted into a desired tissue in an animal. The vectors can also be introduced into embryonic stem cells which are used to generate a transgenic or chimeric (including mosaic) animal. The vectors can also be injected into a fertilized oocyte which is then developed into a transgenic animal.

The reporter gene can be introduced into a transgenic animal containing the neoplastic transformation-promoting genetic modification(s) or into cells in a chimeric animal that contain the neoplastic transformation-promoting genetic mutation. Alternatively, an animal containing the reporter gene can be bred with an animal containing the genetic modification(s) to obtain progeny that contains both the reporter gene and the genetic mutation.

An increase in expression of the reporter gene can be detected, e.g., by measuring the intensity of light emission from a light-emitting product encoded by the reporter gene. Light emission can be measured by conventional techniques, e.g., through use of a luminometer, photometer, camera or other photon detecting device.

Nonhuman Mammals

The present invention includes a nonhuman mammal that contains a cell that contains a recombinant gene construct containing a reporter gene operably linked to a promoter containing a transcriptional regulatory element that is up-regulated by a transcription factor preferentially produced in neoplastic cells. The nonhuman mammal can be transgenic mouse model of cancer. Such models are known in the art. See, e.g., Leder et al, U.S. Pat. No. 4,736,866 and DePinho et al., U.S. Pat. No. 6,639,121. The present invention also can be incorporated into a chimeric nonhuman mammal, e.g., a mouse.

A chimeric nonhuman mammal useful in practicing the invention can be generated by introducing ES cells containing into a host embryo. This can be done, for example, by blastocyst injection or aggregation with earlier stage pre-implantation embryos (e.g., eight-cell embryo). The embryo is subsequently transferred into a surrogate mother for gestation. Chimerism in the born animal can be determined by phenotype (such as fur color, if the host embryo and the ES cells are derived from animal strains of different fur colors), PCR, Southern blot analysis, or biochemical or molecular analysis of polymorphic genes (such as glucose phosphate isomerase). To facilitate identification of chimeric animals having a desired genetic alteration, one can co-introduce a detectable reporter gene and the desired genetic alteration into the ES cells.

Any suitable ES cell line can be used for producing a chimeric nonhuman mammal useful in practicing the invention. Exemplary mouse ES cell lines include, e.g., E14.1, WW6, CCE, J1, and AB1. See also Alex Joyner, Ed., Gene Targeting, A Practical Approach, Chapter 4 (Virginia Papaioannou), Oxford Press, 2^{nd} Ed., (2000). In some embodiments, the ES cell lines provide 10% or higher chimerism. In some embodiments, the ES cell lines provide 90% or higher chimerism.

To increase the contribution of introduced ES cells to a specific tissue, one can use a host embryo that is deficient in generating that issue. This can be accomplished by any suitable method, including inducible expression of a toxin gene, e.g., diphtheria toxin, in a specific cell type, or tissue-specific deletion of genes needed for generating this cell type. In such a complementation system, all or most of the cells of the desired cell type or tissue will be derived from the introduced ES cells.

The use of a chimeric nonhuman mammal provides flexibility in developing models of different diseases. For example, ES cell lines can be established for different cancer models by knocking out a tumor suppressor gene (e.g., p53) and introducing a reporter gene (e.g., luciferase), a tissue- specific reverse tetracycline transactivator gene (i.e., MMTV-rtTA) and an oncogene of choice (e.g., Akt, Her2V664E, Her2, Bcl2, K-Ras and Cyclin D1) under the control of a promoter regulated by reverse tetracycline transactivator (rtTA). These cancer models allow the comparison study of cancers of different etiology, and comparison study of different oncogenes in cancer development.

In some embodiments of the invention, the nonhuman mammals are immunocompromised or immunodeficient. Diseases may develop sooner and/or faster in such animals. To develop such animals, one can use blastocysts derived from, for example, an X-linked SCID animal, or a RAG1−/− or RAG2−/− animal.

As used herein, “chimeric” means chimeric in terms of ontogeny. Accordingly, a chimeric nonhuman mammal is an animal that has grown, i.e., developed, directly from a multicellular embryo into which at least one genetically modified ES cell has been injected or aggregated. A chimeric nonhuman mammal of the invention is to be distinguished from a morphologically developed animal that has received a xenograft, e.g., an organ graft, a tissue graft, or a tumor graft from another animal.

As used herein, “nonhuman mammal” means any mammal other than a human, e.g. a rat, a mouse, a hamster or a guinea pig.

### IV EXAMPLE

This example describes the use of a reporter gene to study colon cancer in mice by employing in vivo bioluminescence imaging of luciferase expression. The example is intended to illustrate the methods and materials of the present invention. Suitable modifications and adaptations of the described conditions and parameters normally encountered in the art which are obvious to those skilled in the art are within the spirit and scope of the present invention. This example is not to be construed as limiting the scope of the invention in any way.

The mouse used here contains gastrointestinal cells whose APC gene can be conditionally knocked out. These cells contain an inducible RNAi construct that specifically inhibits the expression of the APC gene. (Alternatively, a pair of LoxP sites could be inserted into the endogenous APC gene of these cells. Upon expression of a Cre recombinase in these cells, the APC gene is knocked out.) These cells contain also a reporter gene construct comprising a luciferase-coding sequence linked operably to an artificial Top/Fop promoter (Staal et al., International Immunology 11:312-7 (1999)). Colon cancer is induced in the mouse by inhibiting or inactivating the APC gene in the gastrointestinal tissue. This activates β-catenin previously sequestered by APC. The β-catenin binds to the β-catenin response element, which is part of the Top/Fop promoter. This activates expression of the luciferase, which is detected by conventional methods (Contag and Bachmann, Annual Review of Biomed. Eng. 4:235-60 (2002)). Light emission is observed at a basal level in normal gastrointestinal tissues that have not undergone tumorigenesis, and at an increased level in those that have undergone tumorigenesis.

This mouse model permits longitudinal monitoring of tumor onset, progression, and response to therapy and may be used effectively for testing cancer prevention and treatment strategies based on therapeutics that specifically target the APC pathway.

Other embodiments are within the following claims.

We claim:

1. A gene construct comprising a reporter gene operably linked to a promoter containing a transcriptional regulatory element that is up-regulated by a transcription factor preferentially produced in neoplastic cells.

2. The gene construct of claim 1, wherein the transcriptional regulatory element is selected from the group consisting of a β-catenin response element, an E2F response element, a Forkhead response element, and a Smad-2/Smad-3 response element.

3. The gene construct of claim 1, wherein the reporter gene encodes a protein selected from the group consisting of an enzyme, a bioluminescent protein, and a fluorescent protein.

4. The gene construct of claim 3, wherein the enzyme is selected from the group consisting of β-galactosidase, alkaline phosphatase, and chloramphenicol acetyltransferase.

5. The gene construct of claim 3, wherein the bioluminescent protein is a luciferase.

6. The gene construct of claim 3, wherein the fluorescent protein is selected from the group consisting of green fluorescent protein, yellow fluorescent protein, enhanced yellow fluorescent protein, red fluorescent protein and blue fluorescent protein.

7. A cell comprising the gene construct of any one of claims 1-6.

8. The cell of claim 7, wherein the cell is an embryonic stem cell.

9. A nonhuman mammal comprising the cell of claim 7.

10. The nonhuman mammal of claim 9, wherein the mammal further comprises a neoplastic transformation-promoting genetic modification.

11. A method of detecting a neoplasia a nonhuman mammal, comprising
(a) providing a nonhuman mammal, at least some of whose somatic cells are engineered cells comprising a genome comprising a neoplastic transformation-promoting genetic modification and a reporter gene operably linked to a transcriptional regulatory element wherein the transcriptional regulatory element is up-regulated by a transcription factor preferentially produced in neoplastic cells;
(b) allowing time for neoplastic transformation to occur in at least one of the engineered cells, and
(c) detecting a signal from the reporter gene expressed in the engineered cells.

12. The method of claim 7, wherein the nonhuman mammal is a mouse.
